(19) United States
(12) Patent Application Publication
(10) Pub. No.: US 2009/0041666 A1
(43) Pub. Date: Feb. 12, 2009

(54) Ophthalmic formulations of Amyloid-beta contrast agent and methods of use thereof

(76) Inventors: Lee E. Goldstein, Newton, MA (US); Evan A. Sherr, Ashland, MA (US); Paul D. Hartung, Acton, MA (US); Francis X. Smith, Salem, NH (US); Kevin R. Sullivan, Derry, NH (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/154,226

(22) Filed: May 21, 2008

Related U.S. Application Data
(60) Provisional application No. 60/931,189, filed on May 21, 2007.
(60) Provisional application No. 61/062,170, filed on Jan. 23, 2008.

(51) Int. Cl. A61K 49/00; A61K 31/194; A61P 27/02; A61K 31/216

(52) U.S. Cl. 424/9.1; 514/568; 514/533

(57) ABSTRACT

The invention provides ophthalmic formulations of Amyloid-β contrast agents. Also provided are methods of using such formulations in the diagnosis of Alzheimer's Disease or a predisposition thereto as well as methods for the prognosis of Alzheimer's Disease.

### RELATED APPLICATIONS

This application claims the benefit of U.S. Ser. No. 60/931,189, filed May 21, 2007 and U.S. Ser. No. 61/062,170, filed Jan. 23, 2008. Each of these applications is herein incorporated by reference in its entirety.

### FIELD OF THE INVENTION

This invention relates generally to neurodegenerative disease.

### BACKGROUND

Alzheimer's Disease (“AD”) is a chronically progressive degenerative disorder of aging and is a major contributor to morbidity and modality in the elderly. AD currently accounts for about 70% of all cases of dementia and affects some 2-4 million Americans. As many as 9 million Americans may have AD by the year 2050. Epidemiological studies have estimated that if the onset of AD could be delayed by 5 years, the incidence and prevalence of AD would be cut in half.

The accumulation of Amyloid-β (“Aβ”) has been implicated in the pathogenesis of Alzheimer's disease. Aβ has also been shown to accumulate in the lens of the eye at levels, which make the detection of Aβ aggregations in the lens a useful method of diagnosing and evaluating Alzheimer's Disease progress.

### SUMMARY OF THE INVENTION

Congo Red- and Chrysamine G-derivatives such Methoxy-X04 and X34 have previously been used as in vivo contrast agents to detect amyloid-β plaques in the brain. These compounds are not suitable for administration to the eye because of their bioavailabilty, solubility, and/or toxicity characteristics. Accordingly, in order to successfully use these derivative compounds as in vivo contrast agents for detection of Aβ accumulation in the lens of the eye, the compounds are incorporated into ophthalmic formulations having improved bioavailabilty characteristics, while still retaining the Aβ-binding characteristics of the derivatives.

For example, ophthalmic formulations contain an effective amount of a compound of Formula I along with a pharmaceutically acceptable carrier or excipient. Those skilled in the art will recognize that, in Formula I, R′₂ can be selected from the group consisting of H, OH, and OCH₃; R₃ can be selected from the group consisting of H, COOH, and CO₂CH₃; and R₄ can be selected from the group consisting of H, OH, and OCH₃. These ophthalmic formulations are designed to be applied to the cornea and diffuse through the cornea and the aqueous humor to the lens of the eye. Moreover, these ophthalmic formulations are soluble in the cornea, aqueous humor, and lens of the eye. Such formulations preferably have an octanol-water partition coefficient K_{ow} of between 100 and 300 or a LogD value of between 1 and 3. For example, the K_{ow} is 100, 125, 150, 175, 200, 225, 250, 275 or 300 or LogD value is 1, 1.25, 1.50, 1.75, 2, 2.25, 2.5, 2.75, or 3. Preferably, the K_{ow} is between 200 and 300. Suitable compounds include, but are not limited to, the compounds of Formula II, Formula III, Formula VIII, and/or Formula X.

The formulation may also contain a preservative, such as, for example, propyl paraben or benzalkonium chloride, which, when present, is optimally added in a concentration of less than 1%. Moreover, the ophthalmic formulation may also contain a pupil dilating agent (i.e., a mydriatic, such as atropine) in order to provide an optimal field of view for the associated eye test. When the formulation includes the compound of Formula X, the compound of Formula X contains particles less than 6 μm in size (i.e., less than 5 μm, less than 4 μm, less than 3 μm, less than 2 μm, less than 1 μm).

Some Congo Red- and/or Chrysamine G-derivatives are more hydrophobic (and, thus, less water soluble) than others. Therefore, the design of ophthalmic formulations containing this type of derivative compound must take this relative hydrophobicity into account. Ophthalmic formulations containing this class of compounds may be in the form of an ointment containing an effective amount of a compound of Formula I along with a pharmaceutically acceptable carrier or excipient. Those skilled in the art will recognize that, in Formula I, R′₂ can be selected from the group consisting of H, OH, and OCH₃; R₃ can be selected from the group consisting of H, COOH, and CO₂CH₃; and R₄ can be selected from the group consisting of H, OH, and OCH₃. Such ointments preferably have a logP_{oct} value less than 2.6 and are applied to the cornea and diffuse through the cornea and aqueous humor to the lens of the eye. Moreover, these formulations are soluble in the cornea, aqueous humor, and lens of the eye. Exemplary compounds of Formula I for use in such ointments include the compound of Formula VIII (e.g., X04) and the compound of Formula X (e.g., Methoxy-X04). Preferably, the hydrophobic compound of Formula I is the compound of Formula X. When the formulation includes the compound of Formula X, the compound of Formula X contains particles less than 6 μm in size (i.e., less than 5 μm, less than 4 μm, less than 3 μm, less than 2 μm, less than 1 μm).

The excipient used in the preparation of the ointment includes petrolatum, mineral oil, or combinations thereof. For example, one such suitable ophthalmic formulation contains 1% or less of the hydrophobic compound of Formula I, 85% petrolatum, and 15% mineral oil.

Optionally, the ointment may also contain a preservative, such as, for example, propyl paraben or benzalkonium chloride. When present, the preservative is included in a concentration of less than 1%. Likewise, the ointment may further optionally contain a pupil dilating agent, for example a mydriatic such as atropine.

Alternatively, the excipient used with these relatively hydrophobic compounds is an aqueous solution comprising a viscosity agent or an emulsifier. For example, the viscosity agent is hydroxypropyl methyl cellulose. Such formulations contain less than about 1% of a preservative selected from the group consisting of propyl paraben or benzalkonium chloride; and may also optionally contain a pupil dilating agent (e.g., a mydriatic such as, for example, atropine).

For example, one such aqueous solution formulation contains 1% or less of the compound of Formula I; surfactant such as polysorbate 80; a preservative such as benzalkonium chloride; a tonicity agent such as sodium chloride; a buffer such as boric acid or a salt thereof; a chelating agent such as edentate disodium; and a viscosity agent such as hydroxypropyl methylcellulose. The pH is adjusted with acids or bases, such as hydrochloric acid or sodium hydroxide such that the tonicity of the formulation is isotonic relative to the tissue of the eye, thereby causing little or no swelling or contraction of the target tissue. Alternative tonicity agents include boric acid, sodium bicarbonate, and sodium chloride. Moreover, use of an isotonic formulation also results in little or no discomfort upon contact of the eye.

Some Congo Red- and Chrysamine G-derivatives such as X34 are relatively more hydrophilic in nature compared to either the parent compounds from which they are derivated or to the other CR- and CG-derivatives. Ophthalmic formulations containing such derivative compounds are preferably in the form of aqueous solutions containing an effective amount of a compound of Formula I and a pharmaceutically acceptable carrier or excipient and have a LogD value less than 0.42. Those skilled in the art will recognize that, in Formula I, R′₂ can be selected from the group consisting of H, OH, and OCH₃; R₃ can be selected from the group consisting of H, COOH, and CO₂CH₃; and R₄ can be selected from the group consisting of H, OH, and OCH₃. These formulations are designed to be applied to the cornea and are able to diffuse through the cornea and aqueous humor to the lens of the eye. Moreover, these formulations are soluble in the cornea, aqueous humor, and lens of the eye. Suitable compounds for use in such ophthalmic formulations include, for example, the compound of Formula II (e.g. X34) and the compound of Formula III (e.g. Methoxy-X34). Preferably, the compound of Formula I is the compound of Formula II.

Such aqueous solution ophthalmic formulations may contain a preservative (e.g., less than about 1% of propyl paraben or benzalkonium chloride). In addition, these formulations may also contain a pupil dilating agent. For example, the pupil dilating agent may be a mydriatic, such as, for example, atropine.

In some embodiments, these aqueous solution formulations contain a buffered aqueous excipient. By way of non-limiting example, the buffered aqueous excipient is water, propylene glycol, or both. The presence of the buffer provides proper pH for maximum solubility of the compound of Formula I, and the formulation may also contain a chelating agent to improve stability as well as a preservative. Specifically, the buffer is, for example, Tris, the chelating agent is, for example, ethylenediamine-tetraacetate, and the preservative is, for example, parabens. For example, one preferred aqueous solution formulation described herein may contain 1% or less of the compound of Formula I; a solvent such as water; 0.001% to 10% (e.g., 0.001%, 0.005%, 0.010%, 0.05%, 0.1%, 0.5%, 1%, 2.5%, 5%, 7.5%, or 10%) Tris-buffer; 0.001% to 1% (e.g., 0.001%, 0.005%, 0.010%, 0.025%, 0.05%, 0.1%, 0.5%, 0.75%, or 1%) EDTA; and 0.0001% to 1% (e.g., 0.0001%, 0.0005%, 0.001%, 0.005%, 0.01%, 0.05%, 0.1%, 0.11%, 0.5%, or 1%) parabens.

These aqueous solution formulations optionally contain a thickening agent to improve the ease of administration, to improve drug residence time in the eye, or both. By way of non-limiting example, the thickening agent can a cellulose derivative thickening agent such as hydroxypropyl methylcellulose, methylcellulose, or hydroxyethyl cellulose or a non-cellulose thickening agents such as polyvinyl pyrrolidone, polyacrylates, or carbomes. Those skilled in the art will recognize that the thickening agent can be used to increase the viscosity of the formulation up to 1,000,000 centiPoise. Preferably, the viscosity is increased up to 10 centipoise, up to 20 centiPoise, up to 30 centipoise, up to 40 centiPoise, up to 50 centipoise, up to 100 centiPoise, up to 250 centiPoise, up to 500 centipoise, up to 750 centiPoise, or up to 1000 centiPoise. In one preferred embodiment, the viscosity is increased to between 10 and 1000 centiPoise (i.e., to 10, 20, 25, 30, 40, 50, 75, 100, 250, 500, 750, or 1000 centiPoise).

Ophthalmic formulations containing the Congo Red- and Chrysamine G-derivatives may contain less than about 2%, less than about 1.5%, less than about 1%, or less than about 0.5% of a compound of Formula I along with a pharmaceutically acceptable carrier. Those skilled in the art will recognize that, in Formula I, R′₂ can be selected from the group consisting of H, OH, and OCH₃; R₃ can be selected from the group consisting of H, COOH, and CO₂CH₃; and R₄ can be selected from the group consisting of H, OH, and OCH₃. For example, such a formulation will contain less than about 0.1% of the compound of Formula I. Suitable compounds of Formula I that are used in such ophthalmic formulations include, for example, the compounds of Formula II, Formula III, Formula VIII, and/or Formula X. When the formulation includes the compound of Formula X, the compound of Formula X contains particles less than 6 μm in size (i.e., less than 5 μm, less than 4 μm, less than 3 μm, less than 2 μm, less than 1 μm).

For optimal bioavailability for use as an in vivo ocular contrast agent, these formulations have an octanol-water partition coefficient K_{ow} of between 100 and 300 or a LogD value of between 1 and 3 and are designed to be applied to the cornea and diffuse through the cornea and the aqueous humor to the lens of the eye. For example, the K_{ow} is 100, 125, 150, 175, 200, 225, 250, 275 or 300 or LogD value is 1, 1.25, 1.50, 1.75, 2, 2.25, 2.5, 2.75, or 3. Moreover, these formulations are soluble in the cornea, aqueous humor, and lens of the eye. Preferably, the formulations are in the form of a tape, an ointment, an eye drop, or an aqueous solution.

These ophthalmic formulations can also contain a preservative, such as, for example, propyl paraben or benzalkonium chloride. When added to the ophthalmic formulation, the preservative is typically present in a concentration of less than 1%. The inclusion of a pupil dilating agent (e.g., a mydriatic such as atropine) can provide an optimal field of view for the associated eye test.

Also provided herein are methods of diagnosing Alzheimer's Disease or a predisposition thereto in a mammal using any of the ophthalmic formulations disclosed herein. Specifically, an ocular tissue (e.g., a deep cortical region, a supranuclear region, or an aqueous humor region of an eye) is contacted with the ophthalmic formulation, which is allowed to distribute into the lens. Those skilled in the art will recognize that any suitable method(s) of administration or application of the ophthalmic formulations of the invention (e.g., topical, injection, parenteral, airborne, oral, and/or suppository administration, etc.) can be employed. For example, the contacting may occur via topical administration or via injection. Ocular tissue is then imaged using any imaging technique known to those in the art. An increase in binding of the ophthalmic formulation to the ocular tissue compared to a normal control level of binding indicates that the mammal is suffering from or is at risk of developing Alzheimer's Disease.

For example, the increase may be at least 10% greater, at least 25% greater, at least 50% greater, at least 100%, 3-fold, 5-fold, 10-fold, or more greater than said normal control value.

The invention also encompasses a method for prognosis of Alzheimer's Disease by contacting an ocular tissue of a mammal with any of the ophthalmic formulations described herein; allowing the formulation to distribute into the lens; imaging the ocular tissue; quantitating the level of association of the formulation with the ocular tissue; and comparing this level of association with a normal control level of association. Increasing levels of association over time indicates an adverse Alzheimer's Disease prognosis. Those skilled in the art will recognize that any suitable method(s) of administration or application of the ophthalmic formulations of the invention (e.g., topical, injection, parenteral, airborne, oral, and/or suppository administration, etc.) can be employed. For example, the contacting may occur via topical administration or via injection.

The methods are also useful to monitor the effect of a therapeutic intervention; a decrease in the level of association indicates that the intervention is efficacious, i.e., an improvement in disease status, whereas an increase indicates a worsening of the disease or that the intervention is not leading to a measurable clinical benefit.

Likewise, the invention also encompasses methods of diagnosing Alzheimer's Disease or a predisposition thereto in a mammal by administering any of the formulations of the invention containing the compound of Formula X, wherein the particles of the compound of Formula X are less than 6 μm in size of any one of claims to the mammal; allowing the formulation to distribute into the lens of the eye; and imaging an ocular tissue (e.g., a cortical region, a supranuclear region, or an aqueous humor region of an eye), wherein an increase in binding of the formulation to the ocular tissue compared to a normal control level of binding indicates that the mammal is suffering from or is at risk of developing Alzheimer's Disease.

For example, the increase may be at least 10% greater, at least 25% greater, at least 50% greater, or at least 100% greater than said normal control value. Those skilled in the art will recognize that such a formulation can be administered systemically (i.e., via system injection) or ocularly (i.e., via ocular injection).

The invention also provides methods of determining the level of binding of any of the ophthalmic formulations of the invention to ocular tissue, by imaging the ocular tissue after the ophthalmic formulation has been administered and allowed to distribute into the lens of the eye, and comparing the level of binding of said formulation to the ocular tissue to a normal control level of binding.

Finally, also provided is a method of generating a diagnostic index for predicting the development and/or progression of a disease or disorder (e.g., Alzheimer's Disease). For example, the diagnostic index may be generated by collecting a representative number of values or data points such that a determination as to illness or wellness can be made for a given patient. The invention also encompasses the resultant diagnostic index (e.g., the plurality or collection of values that is obtained).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

### DETAILED DESCRIPTION OF THE INVENTION

This invention provides for sensitive means to non-invasively, safely, and reliably detect a biomarker of Alzheimer's Disease in the lens and other ocular tissues using a quasi-elastic light scattering, Raman spectroscopy, fluorometric or any other suitable optical technologies. These techniques allow detection and monitoring of amyloid protein deposition in the eye for the diagnosis of neurodegenerative disorders such as AD and prionopathies. Lens protein aggregation is potentiated by human Aβ₁₋₄₂ peptide, a pathogenic and neurotoxic peptide species which aggregates and accumulates in AD brain. Aβ was found to promote protein aggregation both in vivo and in vitro, and Aβ₁₋₄₂ was found specifically in the deep cortex and supranucleus of human lenses and was associated with large molecular weight protein aggregates. The results indicate that the protein aggregation in the lens, e.g., in lens cortical fiber cells, represents an easily accessible peripheral marker of AD pathology in the brain. (See, U.S. Pat. No. 7,107,092, which is herein incorporated by reference in its entirety).

Methods of diagnosing, prognosing, staging, and/or monitoring mammalian amyloidogenic disorders or predisposition thereto are carried out by detecting a protein or polypeptide aggregate in the cortical and/or supranuclear region of an ocular lens of the mammal. This determination is compared to or normalized against the same determinations in the nuclear region of the same lens where more general effects of aging are observed. Comparisons are also made to a population norm, e.g., data compiled from a pool of subjects with and without disease. The presence of or an increase in the amount of aggregate in the supranuclear and/or cortical lens regions of the test mammal compared to a normal control value indicates that the test mammal is suffering from, or is at risk of, developing an amyloidogenic disorder. A normal control value corresponds to a value derived from testing an age-matched individual known to not have an amyloidogenic disorder or a value derived from a pool of normal, healthy (e.g. non-AD) individuals.

As used herein an “amyloidogenic disorder” is one that is characterized by deposition or accumulation of an amyloid protein or fragment thereof in the brain of an individual. Amyloidogenic disorders include, for example, AD, Familial AD, Sporadic AD, Creutzfeldt-Jakob disease, variant Creutzfeldt-Jakob disease, spongiform encephalopathies, Prion diseases (including scrapie, bovine spongiform encephalopathy, and other veterinary prionopathies), Parkinson's disease, Huntington's disease (and trinucleotide repeat diseases), amyotrophic lateral sclerosis, Downs Syndrome (Trisomy 21), Pick's Disease (Frontotemporal Dementia), Lewy Body Disease, neurodegeneration with brain iron accumulation (Hallervorden-Spatz Disease), synucleinopathies (including Parkinson's disease, multiple system atrophy, dementia with Lewy Bodies, and others), neuronal intranuclear inclusion disease, tauopathies (including progressive supranuclear palsy, corticobasal degeneration, hereditary frontotemporal dementia (with or without Parkinsonism), and Guam amyotrophic lateral sclerosis/parkinsonism dementia complex). These disorders may occur alone or in various combinations. For example, individuals with AD are characterized by extensive accumulation of amyloid in the brain in the form of senile plaques, which contain a core of amyloid fibrils surrounded by dystrophic neurites. Some of these patients also exhibit clinical signs and symptoms, as well as neuropathological hallmarks, of Lewy Body disease.

The presence of and/or an increase in the amount of an amyloid protein or polypeptide detected in a subject's eye tissue over time indicates a poor prognosis for disease, whereas absence or a decrease over time indicates a more favorable prognosis. For example, a decrease in the amount or a decrease in the rate of accumulation in amyloid protein or similar changes in the associated ocular morphological features in eye tissue after therapeutic intervention indicates that the therapy has clinical benefit. Therapeutic intervention includes drug therapy such as, for example, administration of a secretase inhibitor, vaccine, antioxidant, anti-inflammatory, metal chelator, or hormone replacement or non-drug therapies.

Mammals to be tested include human patients, companion animals such as dogs and cats, and livestock such as cows, sheep, pigs, horses and others. For example, the methods are useful to non-invasively detect bovine spongiform encephalopathy (mad cow disease), scrapie (sheep), and other prionopathies of veterinary interests.

For example, the diagnostic test is administered to a human who has a positive family history of familial AD or other risks factors for AD (such as advanced age) or is suspected of suffering from an amyloidogenic disorder, e.g., by exhibiting impaired cognitive function, or is at risk of developing such a disorder. Subjects at risk of developing such a disorder include elderly patients, those who exhibit dementia or other disorders of thought or intellect, as well as patients with a genetic risk factor.

A disease state is indicated by the presence of amyloid protein aggregates or deposits in the supranuclear or deep cortical regions of a mammalian lens. For example, the amount of amyloid protein aggregates is increased in a disease state compared to a normal control amount, i.e., an amount associated with a nondiseased individual. Amyloid proteins include, for example, β amyloid precursor protein (APP), Aβ, or a fragment thereof (e.g., Aβ₁₋₄₂), as well as prion proteins, and synuclein. Protein or polypeptide aggregates may contain other proteins in addition to Aβ (such as α, β, and or γ crystallin). Unlike amyloid protein deposition in brain tissue, which is primarily extracellular, ocular deposition in lens cortical fiber cells is cytosolic.

### Amyloid Imaging Agents

Congo Red (“CR”) is an amyloid-staining agent that is widely used in post-mortem histological identification, evaluation, and/or diagnosis of Alzheimer's Disease. (See Mathis et al., Current Pharmaceutical Design 10(13):1469-92 (2004), incorporated herein by reference in its entirety). CR selectively binds to Aβ aggregations with high affinity. Unfortunately, CR does not have adequate bioavailability characteristics, which makes it unsuitable for use as an in vivo contrast agent.

Chrysamine G (“CG”) is a carboxylic acid analogue of CR that was developed to address (and overcome) some of these shortcomings of CR. Chrysamine G is considerably more lipophilic than Congo Red. Thus, it can cross the blood-brain barrier, and it is useful as a probe for detecting senile plaques (Aβ aggregates) in the brain.

Those skilled in the art will recognize that the hydrophobicity and hydrophilicity of a substance can be measured using the octanol-water partition coefficient (“P_{oct}” or “K_{ow}”), for compounds whose solubility is not altered by the pH and ionic characteristic of the solute, or LogD value, for compounds whose solubility is altered by the pH and ionic characteristic of the solute. LogD is the logarithm of the distribution coefficient, which is the ratio of the sum of the concentrations of all species of a compound in octanol to the sum of the concentrations of all species of the compound in water.

In these contexts, hydrophobicity is related to factors such as absorption, bioavailabity, hydrophobic drug-receptor interactions, metabolism, and/or toxicity. As used herein, the logP_{oct} is synonymous to logK_{ow}, and both measurements are used interchangeably herein. Likewise, logP_{oct} is a functionally equivalent measure to LogD; both values reflect the degree of solubility of a given compound.

The octanol-water partition coefficient of CR at pH 7.4 is only 0.7 (logP_{oct}=0.18), whereas the P_{oct} of CG is nearly 100-fold higher (P_{oct}=60; logP_{oct}=1.8).

In an effort to develop improved in vivo contrast agents, as early as 1998, Klunk et. al described a class of Congo Red or Chrysamine G derivatives that retain their Aβ-binding characteristics while improving systemic bioavailability. (See Klunk et al., Life Sci. 63(20):1807-14 (1998)) The resulting Chrysamine G derivatives are described in U.S. Pat. Nos. 6,133,259; 6,168,776; and 6,114,175, which are herein incorporated by reference in their entireties. See also Mathis et al., Current Pharmaceutical Design 10(13):1469-92 (2004), incorporated herein by reference. These Chrysamine G derivatives exhibit low systemic toxicity as well as modest to poor aqueous solubility (e.g., logP_{oct}=1.8) and modest lipid solubility (e.g., logP_{oct}=1.8).

Examples of such CG derivatives are shown in Table 1. Table 1 also contains a general structure (Formula I) that describes the exemplary CG derivatives. (See Mathos, et al., Current Pharmaceutical Design 10: 1469-92 (2004), herein incorporated by reference).

TABLE 1

| | |
|---|---|
| | (I) |
| 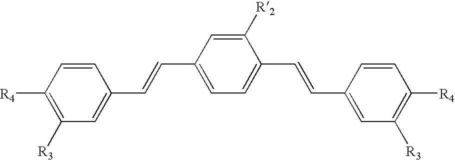 | |

| | 2′-Position | 3-Position | | | | | |
|---|---|---|---|---|---|---|---|
| Compound | (R₂′) | (R₃) | 4-Position (R₄) | Ki (nM) | logPoct | MW | Structure |
| X:E:B34 (X-34) | H | COOH | OH | 18 | 0.42 | 402 | 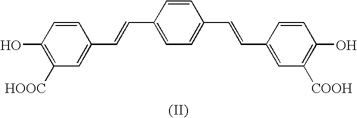 |
| X:E:B34 bis(4-methoxy) | H | CO₂CH₃ | OCH₃ | Inact | 1.2 | 458 | 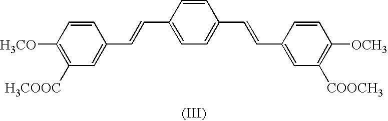 |
| dimethyl ester | | | | | | | |
| Acid Series | | | | | | | |
| X:E:B34 bis(4-methoxy) | H | COOH | OCH₃ | 47 | −0.95 | 430 | 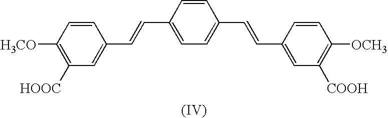 |
| X:E:B30 | H | COOH | H | 135 | 0.39 | 370 | 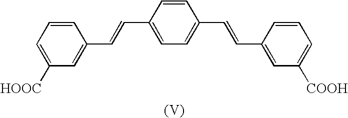 |
| X:E:B30 dimethylester | H | CO₂CH₃ | H | Inact* | 2.5 | 398 | 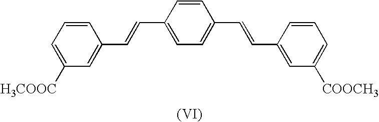 |
| Phenol Series | | | | | | | |
| X:E:B34 dimethylester | H | CO₂CH₃ | OH | 119 | 3.4 | 430 | 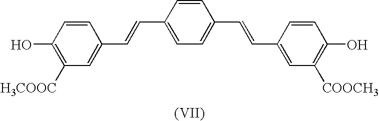 |
| X:E:B04 | H | H | OH | 3100* | 2.0 | 314 | 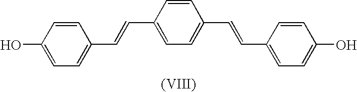 |
| 2′-HO-X:E:B04 | OH | H | OH | 9 | Nd | 330 | 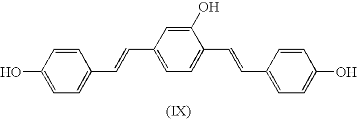 |
| 2-CH3O-X:E:B04 | OCH₃ | H | OH | 27 | 2.6 | 344 | 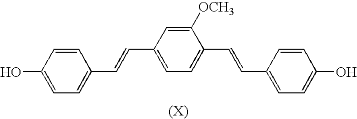 |
| X:E:B04 bis(4-methoxy) | H | H | OCH₃ | Inact* | 2.3 | 342 | 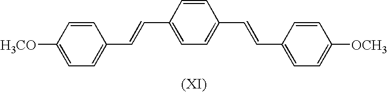 |
| 2′-HO-X:E:B04bis(4-methoxy) | OH | H | OCH₃ | Inact | nd | 358 | 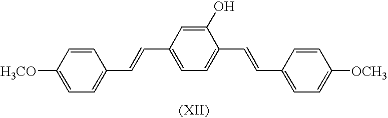 |
| 2′-CH3O-X:E:B04 bis(4-methoxy) | OCH₃ | H | OCH₃ | Inact* | Nd | 372 | 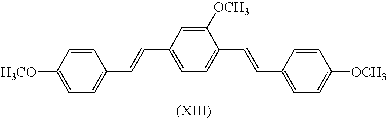 |

*= compound only partially soluble at this concentration
“Inact” = no significant inhibition of [³H] CG binding to Aβ(1-40) at 10 μm
“nd” = logP_{oct} value not determined

Some of these CG derivatives (e.g., Methoxy-X04 (Formula X) and X34 (Formula II)) exhibit native fluorescence, which, when combined with the compounds' high binding efficiency for Aβ aggregations, makes them suitable for use as a fluorescent contrast agents for detection of Aβ aggregations in tissue. Upon binding to Aβ aggregations, these CG derivatives alter the size and mass of the aggregations. Because quasi-elastic light scattering has a theoretical sensitivity of particle radius to the 6^{th} power, binding of these molecules to small beta amyloid aggregations may increase the size of these aggregations, thereby allowing them to reach the sensitivity of detection. Thus, these CG-derivative compounds may also have a role as size-based contrast agents that may be detectable using light scattering techniques such as quasi-elastic light scattering.

The Chrysamine G derivative compounds described herein have previously been used as contrast agents for in vitro and in vivo imaging of Aβ aggregations present in brain tissue. When used to image brain tissue, these derivative compounds are typically provided in injectable form. In fact, both Klunk (Klunk et al., J Neuropath Exp Neurology 61(9):797-805 (2002)), and Goldstein (Moncaster et al., Alzheimer's & Dementia 2(3 Suppl. 1):S51 (2006)) have used these CG derivative imaging agents in injectable formulations as in vivo fluorescent contrast agents. Klunk et al. used these compounds in connection with multi-photon microscopy in order to detect Aβ aggregations in brain tissue of mice. Specifically, Klunk found that doses of 10 mg/kg of Methoxy-X04 (Formula X), when administered via intravenous or intra-peritoneal injection, provided sufficient bioavailability for use as an Aβ-specific contrast agent. Similarly, Goldstein injected comparable levels of Methoxy-X04 (Formula X) into the tail of transgenic 2576 mice and found that the compound could be detected in the supranuclear region of the lens. However, this compound could only be detected using extraordinary ex vivo methods, namely 2-photon fluorescent microscopy at light levels that caused destruction of tissue samples.

Moreover, in both cases, in order to achieve sufficient solubility for systemic injections, the investigators used dimethyl sulfoxide (DMSO) to dissolve Methoxy-X04. However, those skilled in the art will appreciate that DMSO is not a pharmaceutically-acceptable solvent for use in pharmaceutical compositions because of its action as a “carrier” chemical that is able to carry potentially harmful chemicals into the body. Moreover, because of the nature of DMSO solubility, it is difficult to reconstitute dissolved compounds out of DMSO solution.

Likewise, systemic injection of CG derivative compounds for detection of Aβ aggregates in the supranuclear and deep cortical regions of the eye has several critical limitations, e.g., 1) the required doses of contrast agent are very close to the published LD₅₀ for these compounds, which raises the risk of significant systemic toxicity; 2) IV injection of these compounds results in broad systemic distribution and retention, thereby reducing the local bioavailability for a specific target tissue (i.e., the eye); 3) the bioavailability of a systemically introduced contrast agent will be further degraded because there is poor perfusion of the lens of the eye; and 4) the poor solubility parameters of these compounds limits the dosages that can be administered systemically without the use of unacceptable solvents, such as DMSO.

To address these limitations, the invention provides ophthalmic formulations that are more bioavailable to lens tissues, have reduced systemic toxicity, and do not contain solvents that are not suitable for clinical use.

### Ophthalmic Formulations

In order to maximize lens bioavailability, such an ophthalmic formulation exploits the filtering nature of ophthalmic delivery. The eye is a composite structure consisting of alternating hydrophilic (i.e. tear duct and aqueous humor) and hydrophobic (i.e. cornea and lens) layers. Specifically, the cornea, which transmits and focuses light into the eye, is mainly comprised of collagen and lipid molecules. Behind the cornea is the aqueous humor, which is a thin, watery fluid that fills the anterior and posterior chamber of the eye and provides nutrients to the lens and cornea epithelium. The aqueous humor is predominantly comprised of water (>90%), while the lens of the eye is similar to the cornea in structure (e.g., it is mainly comprised of lipid molecules).

Thus, to successfully traverse the solubility transitions found within the cornea, aqueous humor and lens of the eye, the ophthalmic formulations of the invention contains solvents, excipients, and/or carriers that help to balance the intrinsic lipophilicity of the Congo Red or Chrysamine G derivatives. Use of appropriate solvent(s) excipient(s), and/or carrier(s) mediates transit through diverse microenvironments of the eye, thereby permitting these contrast agents to reach the lens, where they will be available to bind to Aβ peptide aggregations present in the supranuclear and/or deep cortical regions.

The CR- or CG-derivative compounds described herein (also referred to herein as “active compounds”), are incorporated into ophthalmic formulations that are suitable for administration to the eye. Such formulations typically comprise the active compound and one or more pharmaceutically-acceptable carriers, excipients and/or solvents. As used herein, “pharmaceutically-acceptable carrier” or “pharmaceutically-acceptable excipient” or “pharmaceutically-acceptable solvent” is intended to include any and all solvents, dispersion media, coatings, antibacterial and antifungal agents, isotonic and absorption delaying agents, and the like, that are compatible with pharmaceutical administration. Suitable carriers are described in the most recent edition of Remington's Pharmaceutical Sciences, a standard reference text in the field, which is incorporated herein by reference. The use of such media and agents for pharmaceutically active substances is well known in the art. Except insofar as any conventional media or agent is incompatible with the active compounds, use thereof in the ophthalmic formulations described herein is contemplated. Supplementary active compounds can also be incorporated into the formulations, as needed.

The ophthalmic formulations of the invention are formulated to be compatible with the intended route of administration (i.e., ocular administration). Solutions or suspensions used for ocular application can include any of the following components: a sterile diluent such as water, saline solution, fixed oils, petrolatum, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and/or agents for the adjustment of tonicity such as sodium chloride or dextrose. The pH can be adjusted with acids or bases, such as hydrochloric acid or sodium hydroxide. Typically, the pH of the formulation will be between 6 and 8. A pH of 7.4 is the most preferred as the native pH of the tear film for ophthalmic formulations.

In all cases, the formulations are sterile, and they are stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper viscosity fluidity is maintained, for example, by the use of a thickening agent such as hydroxypropyl methyl cellulose. Prevention of the action of microorganisms is achieved by various antibacterial and antifungal agents, for example, parabens, chlorobutanol, phenol, ascorbic acid, thimerosal, and the like.

For ocular administration, the active compounds are formulated into ointments (e.g., tapes), salves, gels, aqueous solutions, eye drops, or creams, using procedures and methods generally known in the art. To maximize transit of these ophthalmic formulations to the lens, a pupilary dilating agent can also be used. For example, dilation of the pupil is achieved using a mydriatic. Examples of suitable mydriatics include, but are not limited to atropine, cocaine, tropicamide, cyclopentolate, homatropine, tropicamide, oxyphenonium bromide, lachesine chloride, scopolamine (for short duration dilation), or any other appropriate drug. The use of a pupil dilating agent helps to improve optical access with the convenience of a single administration of the ophthalmic formulation of the invention containing both the desired contrast agent and mydriatic.

Any of the ophthalmic formulations described herein can be included in a container, pack, or dispenser together with instructions for administration.

Dosages

In any of the ophthalmic formulations described herein, the CR or CG derivative compound will be present in the formulation in a concentration less than 2%. For example, the formulation may contain less than 1%, less than 0.5%, less than 0.25%, less than 0.10%, or less than 0.05% of the derivative compound. One preferred formulation will contain less than 0.1% of the active compound (e.g., 1 mg per gm of carrier). Those skilled in the art will recognize the use of a formulation having less than 0.1% of the CR or CG derivative is preferred for a variety of reasons including, but not limited to, regulatory approval, maintenance of the compound in solution, and/or cost.

When a CG derivative compound (e.g., MeX04) is formulated for use as an in vivo contrast agent to detect Aβ plaques within the brain, typical doses administered may be either 10 mg/gm or 10 mg/kg. (See Klunk et al., J. Neuropathol Exp. Neurol 61:797-805 (2002)). For ophthalmic uses, such as those contemplated herein, the total dose of the derivative compound to be administered is unlikely to exceed 1 mg per administration due to solubility and utility limitations. Because the formulations are applied topically, there is greater bioavailability in the region of interest. Moreover, due to the limited solubility of the compounds, topical application is largely independent of subject body weight as there is little risk of system affect at the doses used. Thus, for an average 70 kg patient, the total ophthalmic dosage administered would be approximately a 14 mg/kg systemic dose, which is nearly an order of magnitude less than the dosages administered in the prior art.

Those skilled in the art will recognize that each Congo Red or Chrysamine G derivative described herein has its own unique solubility characteristics. Thus, the choice of the appropriate solvent, excipient, and/or carrier will depend upon the characteristics of the particular CR or CG derivative to be used in the manufacture of a given ophthalmic formulation. Two illustrative examples are presented herein: (i) the compound of Formula X known as Methoxy-X04 (also referred to herein as “MeX04”), which is a hydrophobic example and (ii) the compound of Formula II, known as X34, which is a hydrophilic example. (See Table 1, supra). However, other members of this class of compounds may also be considered and employed in the ophthalmic formulations described herein. In addition, each CR or CG derivative will likely require compound specific carriers and/or excipients in order to prepare an ophthalmic formulations having improved performance characteristics for use as in vivo contrast agents. Determination of the appropriate carriers, solvent and/or excipients for a given CR or CG derivative is within the routine level of skill in the relevant art.

Methoxy-X04

Methoxy-X04 (Formula X) is a fourth generation ligand molecule derived from Congo Red. It is a compound exhibiting low toxicity (Oral rat LD₅₀ of ˜15 g/kg). At room temperature, MeX04 is a yellow powder, which fluoresces upon exposure to UV light. The logP_{oct} for MeX04 is 2.6. While Methoxy-X04 is insoluble in water, it does exhibit reasonable lipid solubility, which may assist its diffusion across the cornea to the lens of the eye. The degree of solubility for Methoxy-X04 is characterized by the compound's K_{ow} (or octanol water partition coefficient), which is used to characterize the relative hydrophilic/hydrophobic solubility of compounds whose solubility is not affected by solvent pH or ionic characteristic. Ophthalmic formulations containing Methoxy-X04 having a K_{ow} of between 100 and 300 (preferably greater than 125; more preferably greater than 200) is preferred to traverse the solubility barriers in the eye.

The chemical structure of Methoxy-X04 is shown below (Formula X).

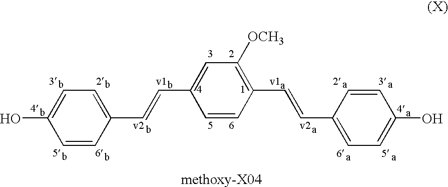

Methoxy-X04 selectively binds to Aβ plaques in both in vivo animal models as well as ex vivo human tissue. This Aβ binding ability, combined with the compound's native fluorescence makes MeX04 a useful in vivo marker for Aβ protein aggregations found in the lens tissue of Alzheimer's patients.

Because these aggregations have been shown to accumulate within the supranuclear and/or deep cortical regions of the eye lens, administration to the surface of the eye is a convenient route of administration for ophthalmic formulations containing this compound. However, because Methoxy-X04 is insoluble in water, an excipient consisting primarily of petrolatum and mineral oil, both United States Pharmacopeia-National Formulary (“USP-NF”) compounds, is a suitable ophthalmic formulation.

Specifically, a mixture of approximately 85% petrolatum and 15% mineral oil is a suitable carrier. At concentrations of 0.1% Methoxy-X04 (e.g., 1 mg active compound per gm of carrier), which is an exemplary dose for the ophthalmic formulations, Methoxy-X04 remains in solution. The petrolatum in this ophthalmic formulation can act as a carrier for a combination of dissolved and suspended Methoxy-X04, by altering the aqueous portions of the eye to accept the compound more readily by adding lipophilic material to the solution of the eye's environment and by altering the interactions of the eye with the compound by shielding it from the aqueous environment. At concentrations of 10 mg/gm of carrier, Methoxy-X04 remains primarily in suspension.

The use of MeX04 at either concentration level (e.g., 1 mg/gm carrier or 10 mg/gm carrier), does not require the use of a preservative because of the anti-microbial nature of petrolatum and mineral oil. Nevertheless, various preservatives, including, but not limited to, propylparaben or benzalkonium chloride, are optionally added as additional preservative to ophthalmic formulations containing MeX04. If added, these preservatives are typically included in concentrations of less than about 1%.

Another method of delivery for hydrophobic compounds such as Methoxy-X04 is to suspended the compound in a vehicle excipient containing a suitable ophthalmic emulsifier, such as, for example, hydroxypropyl methyl cellulose. This use of an emulsifier serves to shield the hydrophobic compound from aqueous environments, thereby maintaining it in a suspension/solution that is capable of traversing diverse environments in the eye.

By way of nonlimiting example, one such aqueous solution formulation contains 1% or less of the compound of Formula I; surfactant such as polysorbate 80; a preservative such as benzalkonium chloride; a tonicity agent such as sodium chloride; a buffer such as boric acid or a salt thereof; a chelating agent such as edentate disodium; and a viscosity agent such as hydroxypropyl methylcellulose. When the ophthalmic formulation comprises ˜0.1% or less of the hydrophobic compound (e.g., Methoxy-X04) as well as ˜0.3% hydroxypropyl methylcellulose, ˜0.1% polysorbate 80, ISSsolution (as a preservative), sterile water, ˜0.4% NaCl, ˜1% boric acid, ˜0.2% sodium borate 10-hydrate, ˜0.03% edetate disodium dihydrate, sodium hydroxide (NaOH) and hydrochloric acid (HCl) are acceptable carriers.

As indicated, at concentrations of 0.1% Methoxy-X04 (e.g., 1-mg active per gm carrier), Methoxy-X04 remains in solution. Hydroxypropyl methylcellulose can act in part as a carrier of microparticles in suspension for a mixture of dissolved and suspended Methoxy-X04. This effect occurs by altering the aqueous portions of the eye to enable them to accept the hydrophobic compounds more readily, by adding lipophilic material to the eye's environment and/or by altering the interactions of the compound with the eye by shielding the hydrophobic compound from the aqueous environment.

When employed in an aqueous formulation, various preservatives, such as, for example, propylparaben or benzalkonium chloride are added. Typically, such preservatives are included in concentrations of less than 1%.

To minimize injury/irritation to the cornea during topical application, particle size in formulation is preferably less than 25 microns in diameter, preferably less than 12 microns, more preferably less than 6 microns in an aqueous solution. If Methoxy-X04 is formulated into a parenteral (injectable) formulation suitable for administration to the eye, typical particle size distribution for safe administration is a fineness of at least 99% less than 10 microns and at least 75% less than 5 microns. To achieve an acceptable particle size, a grinding process is used to insure proper particle size while maximizing yield (i.e., minimizing loss) of material following the grinding process. Those skilled in the art will recognize that any other suitable methods for controlling the particle size known in the art can also be employed. By way of non-limiting example, such methods may include crushing, milling, screening, and/or controlled growth of crystals.

For example, Polysorbate 80 is weighed into a polypropylene bottle, and an initial amount of sterile water for injection, USP, is weighed into the bottle. Next, Methoxy-X04 is weighed and added to the bottle. The current batch weight is determined, and the final amount of sterile water for injection to adjust the formulation to a final batch weight is added. Then, the YTZ grinding media is added to the bottle, and the bottle is placed on a roller mill for a minimum of 12 hours at a setting of 50%.

After the milling process, a polishing filter and Masterflex tubing are connected to the bottom of a Millipore housing assembly, while a high-pressure hose is attached to the top of the Millipore housing. Using a nitrogen pressure gauge, the solution is pushed thorough the housing and the polish filter. For example, the pressure used is between 3 psi and 5 psi. This grinding process yields micronized Methoxy-X04 having a mean particle size of 1.157 μm with 100% of particles smaller than 10 μm and greater than 75% of particles smaller than 5 μm. Thus, those skilled in the art will recognize that this exemplary milling process produces Methoxy-X04 particles that are suitable for either topical or parenteral formulation, without requiring the use of solvents, such as dimethyl sulfoxide, that are incompatible with use in drug formulations.

X34

X34 (Formula II) is a ligand molecule that is derived from Congo Red by replacing the naphthalene sulfonic acids with salicylic acids and the azo linkage with an ethenyl link. X34 is a compound that exhibits low toxicity (Oral rat LD₅₀ of ˜15 g/kg). At room temperature, X34 is a yellow powder, which fluoresces upon exposure to UV light. The logP_{oct} for X34 is 0.42. Moreover, X34 is moderately soluble in water, when buffered to the proper pH. At concentrations of 0.1% of X34 (e.g., 1 mg active per gm of carrier), which is an exemplary clinical dose, X34 appears to remain in solution. Preferably, ophthalmic formulations containing X34 (or other hydrophilic compounds of Formula I) have LogD values of between 1 and 3.

The chemical structure of X34 is provided below.

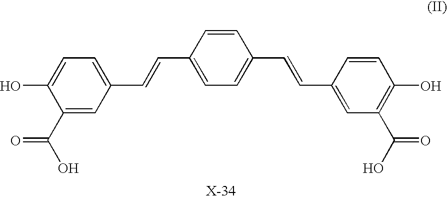

X34 selectively binds to β-amyloid plaques in both in vivo animal models as well as ex vivo human tissue. This fact, combined with the compound's native fluorescence, makes X34 would a useful in vivo marker for β-amyloid aggregations found in the lens tissue of Alzheimer's patients. Because these protein aggregations accumulate in the deep cortical and/or supranuclear regions of the lens of the eye, the eye would be the most convenient route of administration for ophthalmic formulations containing this compound.

A buffered, aqueous excipient is used in the preparation of a suitable ophthalmic formulation containing X34 as the contrast agent. The presence of the buffer provides proper pH for maximum solubility of the compound of Formula I, and the formulation may also contain a chelating agent to improve stability as well as a preservative. Specifically, the buffer is, for example, Tris, the chelating agent is, for example, ethylenediamine-tetraacetate, and the preservative is, for example, parabens. For example, one preferred aqueous solution formulation described herein may contain 1% or less of the compound of Formula I; a solvent such as water; 0.001% to 10% (e.g., 0.001%, 0.005%, 0.010%, 0.05%, 0.1%, 0.5%, 1%, 2.5%, 5%, 7.5%, or 10%) Tris-buffer; 0.001% to 1% (e.g., 0.001%, 0.005%, 0.010%, 0.025%, 0.05%, 0.1%, 0.5%, 0.75%, or 1%) EDTA; and 0.0001% to 1% (e.g., 0.0001%, 0.0005%, 0.001%, 0.005%, 0.01%, 0.05%, 0.1%, 0.11%, 0.5%, or 1%) parabens. In another example, a mixture of approximately 0.1% X34 (as an active ingredient), water (as a solvent), 2% propylene glycol (as a co-solvent for preservative), 0.5% Tris(hydroxymethyl)aminomethane (Tris-buffer) (as a buffer to provide proper pH for maximum solubility of X34), 0.025% ethylenediamine-tetraacetate dihydrate (EDTA Dihydrate) (as a chelating agent to improve stability of formulation), and a total of approximately 0.11% mixed Parabens (as a preservative) are useful excipients.

Various other preservatives, including, but not limited to, for example, benzalkonium chloride or parabens in the X34 ophthalmic formulations described herein. If added, these preservatives are used in the ophthalmic formulations in concentrations of less than 1%.

The use of a thickening agents, including, but not limited to cellulose derivative thickening agents such as hydroxypropyl methylcellulose, methylcellulose, hydroxyethyl cellulose and non-cellulose agents such as polyvinylpyrrolidone, polyacrylates, and carbomes in these X34-containing aqueous ophthalmic formulations improve the ease of administration and/or to improve residence time of the contrast agent within the eye. Using these thickening agents, the viscosity of ophthalmic formulations containing X34 is increased to not more than 1,000,000 centiPoise. Optimally, the viscosity can be increased to approximately 10-1000 centipoise.

Additional Ophthalmic Delivery Vehicles

Other ophthalmic delivery vehicles, such as liposome encapsulated Congo Red or Chrysamine G derivative, micro-encapsulations or other vehicles may be employed to improve transport of Congo Red or Chrysamine G derivatives and the ophthalmic formulations described herein from the cornea through the aqueous humor to the lens of the eye. Electrophoresis, ultrasound phoresis, or other phoretic techniques are optionally used to improve transport to a target anatomical location in the eye.

In some embodiments, the ophthalmic formulations are compounded with carriers that protect the compound against rapid elimination from the body, such as controlled release formulations, including implants, microencapsulated delivery systems, and the like. Biodegradable, biocompatible polymers can also be used, such as, for example, ethylene vinyl acetate, polyanhydrides, polyglycolic acid, collagen, polyorthoesters, and polylactic acid. Methods for preparation of such formulations are known to those skilled in the art. The materials are commercially available from Alza Corporation and Nova Pharmaceuticals, Inc. Liposomal suspensions are useful as pharmaceutically acceptable carriers and can be prepared according to methods known to those skilled in the art, for example, as described in U.S. Pat. No. 4,522,811.

### Ocular Detection of Aβ Protein Aggregates

The ophthalmic formulations described herein are used in conjunction with any optical imaging or detection devices, which collect data from the lens. (See, e.g., U.S. Pat. No. 7,107,092, herein incorporated by reference in its entirety). Aggregates are detected non-invasively, i.e., using a device or apparatus that is not required to physically contact ocular tissue.

The invention includes methods of diagnosing an amyloidogenic disorder or a predisposition thereto in a mammal, by illuminating mammalian lens tissue with an excitation light beam and detecting scattered or other light signals emitted from the tissue that are known in the art. Aggregates are detected with quasi-elastic light scattering techniques (a.k.a. dynamic light scattering), Raman spectroscopy, fluorimetry, and/or other methods of analyzing light returned from the test tissue. An increase of scattered light emitted from the cortical and/or supranuclear regions of an ocular lens indicates that the mammal is suffering from, or is at risk of developing an amyloidogenic disorder such as AD. Excitation light is in the range of 350-850 nm. Preferably, the excitation light beam is a low wattage laser light such as one with a wavelength of 450-550 nm. Those skilled in the art will recognize that it is desirable to choose an excitation light wavelength that avoids lens autofluorescence, which typically emits at wavelengths of about 450 nm. For example, the excitation light beam, which would produce an emission wavelength of approximately 500±20 nm is preferred. Alternatively, the excitation light beam is in the very near-UV (392-400 nm) or visible (400-700 nm) ranges.

Detection of protein aggregation or accumulation or deposition of amyloidogenic proteins or peptides in the supranuclear/cortical region of an ocular lens is ratiometrically, volumetrically, or otherwise mathematically compared to the same or similar measurements in the nuclear or other regions of the lens. These methods are useful to measure protein aggregation or accumulation or deposition of amyloidogenic proteins or peptides in other ocular tissues, including but not limited to the cornea, the aqueous humor, the vitreous humor, the lens, e.g., the supranuclear or deep cortical region of the lens, and the retina.

The QLS technique is used to non-invasively detect and quantitate lens protein aggregation in this animal model of AD and in human subjects. An additional advantage to using this technique is the ability to monitor disease progression as well as responsiveness to therapeutic intervention. Aβ-associated lens aggregates are found exclusively in the cytoplasmic intracellular compartment of human lens cells, specifically lens cortical fiber cells in contrast to Aβ deposits in the brain, which are largely extracellular. Aβ fosters human lens protein to aggregate through metalloprotein redox reactions and this aggregation by chelation or antioxidant scavengers.

The major proteins that can scatter light in a human eye lens are α-, β-, γ-crystallins. Since the crystallins are abundant and large molecules (molecular weight ˜10⁶ Daltons), they induce the greatest amount of scattering of light, including laser radiation in dynamic light scattering (DLS) measurements. When the lens protein molecules are aggregated, they give rise to lens opacities. The lens gradually becomes cloudy as a result of light scattering and absorbance, thereby hindering light transmission and the ability to focus a sharp image on the retina at the back of the eye.

Methods for measuring DLS, are known in the art, e.g., Benedek, G. B., 1997, Invest. Opthalmol. Vis. Sci. 38:1911 1921; Betelhiem, et al., 1999, J. Biochem. Biophys. Res. Comm. 261(2):292 297, Ansari et al., Diabetes Technol. Ther. Summer 1(2): 159-68 (1999); and U.S. Pat. No. 5,540,226. For example, a monochromatic, coherent, low-powered laser is shined into the lens of a subject such as a human patient. Agglomerated particle dispersions within the lens reflect and scatter the light. Light scattering is detected using a variety of known methods such as photo multiplier tube, a solid-state photo diode or a charge coupling device. Because of random, Brownian motion of the lenticular protein crystallins, the concentration of the crystallins appears to fluctuate, and hence, the intensity of the detected light also fluctuates. However, a temporal autocorrelation function of the photo current is mathematically analyzed to reveal the particle diffusivity. The data reveals the composition and extent of cataractogenesis. An increase in light scattering in the supranuclear and/or cortical region of the lens (alone and/or normalized to the scattering in the lens nucleus, where general aging effects on the lens predominate and/or normalized for age) compared to a known normal value or a normal control subject indicates the presence of protein aggregation associated with a neurodegenerative disease such as AD. This finding, in turn, serves as a biomarker for the AD disease process and hence is of clinical utility in the diagnosis, prognosis, staging, and monitoring of the AD or other amyloidogenic disorders.

### EQUIVALENTS

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

We claim:

1. An ophthalmic formulation comprising an effective amount of a compound of Formula I and a pharmaceutically acceptable carrier or excipient, wherein the formulation has an octanol-water partition coefficient K_{ow} of between 100 and 300 or a LogD value of between 1 and 3 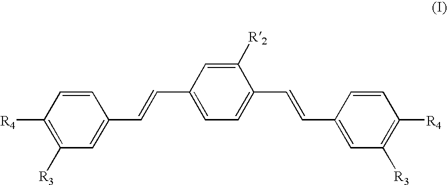
wherein
R′₂ is selected from the group consisting of H, OH, and OCH₃;
is selected from the group consisting of H, COOH, and CO₂CH₃; and
R₄ is selected from the group consisting of H, OH, and OCH₃.

2. The formulation of claim 1, wherein the compound of Formula I is selected from the group consisting of compounds of Formula II, Formula III, Formula VIII, and Formula X.

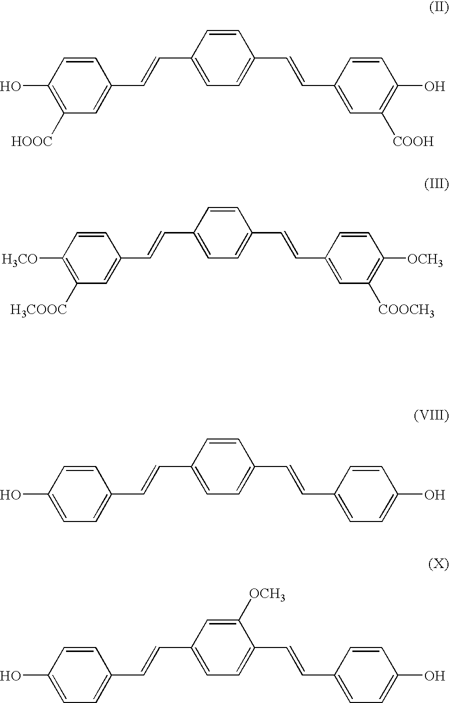

3. The formulation of claim 1, wherein the formulation is soluble in the cornea, aqueous humor, and lens of the eye.

4. The formulation of claim 1, wherein the formulation further comprises a preservative.

5. The formulation of claim 4, wherein the preservative is present in a concentration of less than 1%.

6. The formulation of claim 1, wherein the formulation further comprises a pupil dilating agent.

7. The formulation of claim 6, wherein the pupil dilating agent is a mydriatic.

8. The formulation of claim 7, wherein the mydriatic is atropine.

9. The formulation of claim 1, wherein the K_{ow} is between 200 and 300.

10. The formulation of claim 2, wherein the compound of Formula X comprises particles less than 6 μm in size.

11. An ophthalmic formulation, wherein said formulation is an ointment comprising an effective amount of a compound of Formula I and a pharmaceutically acceptable carrier or excipient, wherein said formulation has a logP_{oct} value less than 2.6 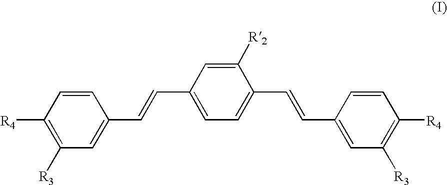
wherein
R′₂ is selected from the group consisting of H, OH, and OCH₃;
R₃ is selected from the group consisting of H, COOH, and CO₂CH₃; and
R₄ is selected from the group consisting of H, OH, and OCH₃.

12. The formulation of claim 11, wherein said formulation is soluble in the cornea, aqueous humor, and lens of the eye.

13. The formulation of claim 11, wherein the compound of Formula I is selected from the group consisting of the compound of Formula VIII and the compound of Formula X.

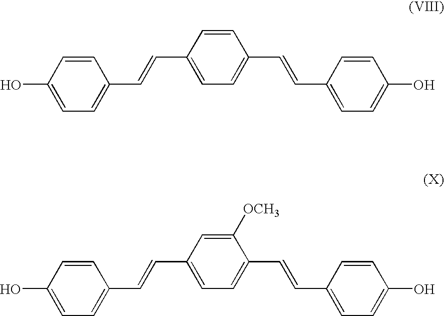

14. The formulation of claim 13, wherein the compound of Formula I is the compound of Formula X.

15. The formulation of claim 11, wherein the excipient is selected from the group consisting of petrolatum, mineral oil, or combinations thereof.

16. The formulation of claim 15, wherein the formulation comprises 1% or less of the hydrophobic compound of Formula I, 85% petrolatum, and 15% mineral oil.

17. The formulation of claim 11, wherein the formulation further comprises a preservative.

18. The formulation of claim 17, wherein the preservative is present in a concentration of less than 1%.

19. The formulation of claim 11, wherein the formulation further comprises a pupil dilating agent.

20. The formulation of claim 19, wherein the pupil dilating agent is a mydriatic.

21. The formulation of claim 20, wherein the mydriatic is atropine.

22. The formulation of claim 11, wherein the excipient is an aqueous solution comprising a viscosity agent.

23. The formulation of claim 22, wherein the formulation comprises:
(a) 1% or less of the compound of Formula I;
(b) a surfactant comprising polysorbate 80;
(c) a preservative comprising benzalkonium chloride;
(d) a tonicity agent comprising sodium chloride;
(e) a buffer comprising boric acid or a salt thereof;
(f) a chelating agent comprising edentate disodium; and
(g) a viscosity agent comprising hydroxypropyl methylcellulose.

24. The formulation of claim 22, wherein the formulation comprises a preservative.

25. The formulation of claim 24, wherein the preservative is present in a concentration of less than 1%.

26. The formulation of claim 22, wherein the formulation further comprises a pupil dilating agent.

27. The formulation of claim 26, wherein the pupil dilating agent is a mydriatic.

28. The formulation of claim 27, wherein the mydriatic is atropine.

29. The formulation of claim 14, wherein the compound of Formula X comprises particles less than 6 μm in size.

30. An ophthalmic formulation, wherein said formulation is an aqueous solution comprising an effective amount of a compound of Formula I and a pharmaceutically acceptable carrier or excipient, wherein said formulation has a LogD value less than 0.42 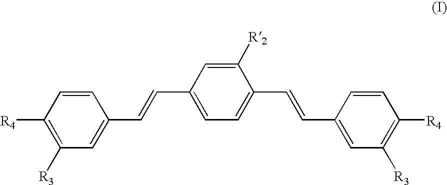
wherein
R′₂ is selected from the group consisting of H, OH, and OCH₃;
R₃ is selected from the group consisting of H, COOH, and CO₂CH₃; and
R₄ is selected from the group consisting of H, OH, and OCH₃.

31. The formulation of claim 30, wherein said formulation is soluble in the cornea, aqueous humor, and lens of the eye.

32. The formulation of claim 30, wherein the compound of Formula I is selected from the group consisting of the compound of Formula II and the compound of Formula III.

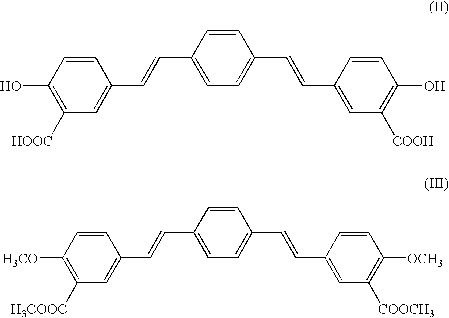

33. The formulation of claim 32, wherein the compound of Formula I is the compound of Formula II.

34. The formulation of claim 30, wherein the formulation comprises a buffered aqueous excipient.

35. The formulation of claim 34, wherein the buffered aqueous excipient comprises water, propylene glycol, or both.

36. The formulation of claim 35, wherein the formulation comprises a buffer to provide proper pH for maximum solubility of said compound of Formula I, a chelating agent, and a preservative.

37. The formulation of claim 36, wherein said buffer is Tris, wherein said chelating agent is ethylenediamine-tetraacetate, and wherein said preservative is parabens.

38. The formulation of claim 36, wherein the formulation comprises
a) 1% or less of the compound of Formula I;
b) a solvent comprising water;
c) 0.001% to 10% Tris-buffer;
d) 0.001% to 1% EDTA; and
e) 0.0001% to 1% parabens.

39. The formulation of claim 36, wherein the preservative is selected from the group consisting of propyl paraben and benzalkonium chloride.

40. The formulation of claim 36, wherein the preservative is present in a concentration of less than 1%.

41. The formulation of claim 30, wherein the formulation further comprises a pupil dilating agent.

42. The formulation of claim 41, wherein the pupil dilating agent is a mydriatic.

43. The formulation of claim 42, wherein the mydriatic is atropine.

44. The formulation of claim 30, wherein the formulation further comprises a thickening agent.

45. The formulation of claim 44, wherein the thickening agent is selected from the group consisting of cellulose derivative thickening agents, hydroxypropyl methylcellulose, methylcellulose, hydroxyethyl cellulose, non-cellulose thickening agents, polyvinyl pyrrolidone, polyacrylates, and carbomes.

46. The formulation of claim 44, wherein the thickening agent increases the viscosity of the formulation up to 1,000,000 centiPoise.

47. The formulation of claim 46, wherein the thickening agent increases the viscosity of the formulation to between 10 and 1000 centipoise.

48. An ophthalmic formulation comprising less than about 2% of a compound of Formula I and a pharmaceutically acceptable carrier. 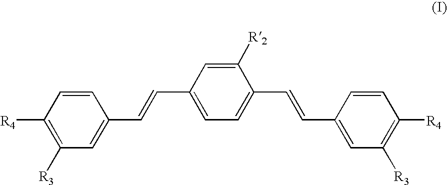
wherein
R′₂ is selected from the group consisting of H, OH, and OCH₃;
R₃ is selected from the group consisting of H, COOH, and CO₂CH₃; and
R₄ is selected from the group consisting of H, OH, and OCH₃.

49. The formulation of claim 48, wherein the formulation comprises less than about 0.1% of the compound of Formula I.

50. The formulation of claim 48, wherein the compound of Formula I is selected from the group consisting of the compounds of Formula II, Formula III, Formula VIII, and Formula X.

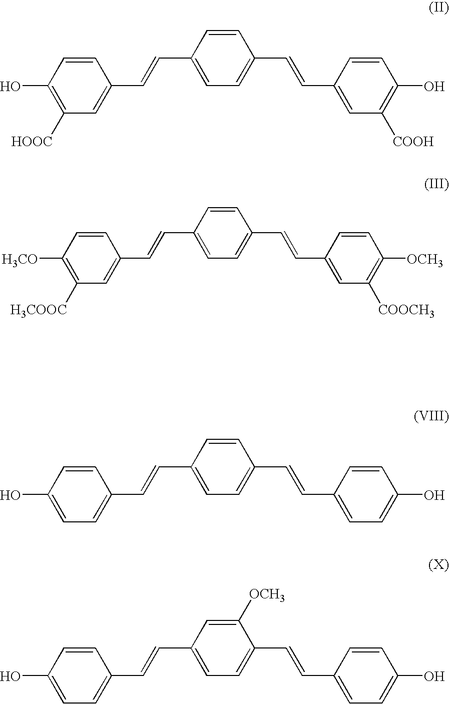

51. The formulation of claim 48, wherein the formulation has an octanol-water partition coefficient K_{ow} of between 100 and 300 or a LogD value of between 1 and 3.

52. The formulation of claim 51, wherein the formulation is soluble in the cornea, aqueous humor, and lens of the eye.

53. The formulation of claim 48, wherein the formulation is in the form of a tape, an ointment, an eye drop, or an aqueous solution.

54. The formulation of claim 48, wherein the formulation further comprises a preservative selected from the group consisting of propyl paraben and benzalkonium chloride.

55. The formulation of claim 54, wherein the preservative is present in a concentration of less than 1%.

56. The formulation of claim 48, wherein the formulation further comprises a pupil dilating agent.

57. The formulation of claim 56, wherein the pupil dilating agent is a mydriatic.

58. The formulation of claim 57, wherein the mydriatic is atropine.

59. The formulation of claim 50, wherein the compound of Formula X comprises particles less than 6 μm in size.

60. A method of diagnosing Alzheimer's Disease or a predisposition thereto in a mammal, comprising
(a) contacting an ocular tissue with the ophthalmic formulation of any one of claims 1, 11, 30, or 48;
(b) allowing said formulation to distribute into the lens; and
(c) imaging said ocular tissue,
wherein an increase in binding of said formulation to said ocular tissue compared to a normal control level of binding indicates that said mammal is suffering from or is at risk of developing Alzheimer's Disease.

61. The method of claim 60, wherein said ocular tissue comprises a cortical region of an eye.

62. The method of claim 60, wherein said ocular tissue comprises a supranuclear region of an eye.

63. The method of claim 60, wherein said ocular tissue comprises an aqueous humor region of an eye.

64. The method of claim 60, wherein said increase is at least 10% greater than said normal control value.

65. The method of claim 60, wherein said increase is at least 25% greater than said normal control value.

66. The method of claim 60, wherein said increase is at least 50% greater than said normal control value.

67. The method of claim 60, wherein said increase is at least 100% greater than said normal control value.

68. The method of claim 60, wherein the formulation is applied to the cornea and is able to diffuse through the cornea and the aqueous humor to the lens of the eye.

69. The method of claim 60, wherein said contacting in step (a) occurs via topical administration of said ophthalmic formulation.

70. The method of claim 60, wherein said contacting in step (a) occurs via injection of said ophthalmic formulation.

71. A method for prognosis of Alzheimer's Disease, comprising
(a) contacting ocular tissue of a mammal with the ophthalmic formulation of any one of claims 1, 11, 30, or 48;
(b) allowing said formulation to distribute into the lens
(c) imaging said ocular tissue;
(d) quantitating the level of association of said formulation with said ocular tissue; and
(e) comparing said level of association with a normal control level of association, wherein increasing levels of association over time indicates an adverse prognosis.

72. A method of diagnosing Alzheimer's Disease or a predisposition thereto in a mammal, comprising
(a) administering the formulation of any one of claims 10, 29, or 59 to the mammal;
(b) allowing said formulation to distribute into the lens of the eye; and
(c) imaging an ocular tissue,
wherein an increase in binding of said formulation to said ocular tissue compared to a normal control level of binding indicates that said mammal is suffering from or is at risk of developing Alzheimer's Disease.

73. The method of claim 72, wherein said ocular tissue comprises a cortical region, a supranuclear region, or an aqueous humor region of an eye.

74. The method of claim 72, wherein said increase is at least 10% greater than said normal control value.

75. The method of claim 72, wherein said increase is at least 25% greater than said normal control value.

76. The method of claim 72, wherein said increase is at least 50% greater than said normal control value.

77. The method of claim 72, wherein said increase is at least 100% greater than said normal control value.

78. The method of claim 72, wherein the formulation is administered systemically.

79. The method of claim 78, wherein the formulation is administered via systemic injection.

80. The method of claim 72, wherein the formulation is administered ocularly.

81. The method of claim 80, wherein the formulation is administered via ocular injection.
